(19) United States
(12) Plant Patent Application Publication
(10) Pub. No.: US 2006/0015978 P1
(43) Pub. Date: Jan. 19, 2006

(54) Osteospermum plant named 'Oste Pur'

(50) OSTEOSPERMUM ECKLONIS; OSTEOSPERMUM

(75) Inventor: Pim Kaagman, PE Andjik (NL)

Correspondence Address: JONDLE & ASSOCIATES P.C., 858 HAPPY CANYON ROAD SUITE 230, CASTLE ROCK, CO, 80108, US

(73) Assignee: Goldsmith Seeds, Inc.

(21) Appl. No.: 10/891,244

(22) Filed: Jul. 14, 2004

(51) Int. Cl. A01H 9/00

(52) U.S. Cl. PLT/379

(57) ABSTRACT

An Osteospermum cultivar particularly distinguished by light violet colored flowers, dark green foliage and vigorous growth.

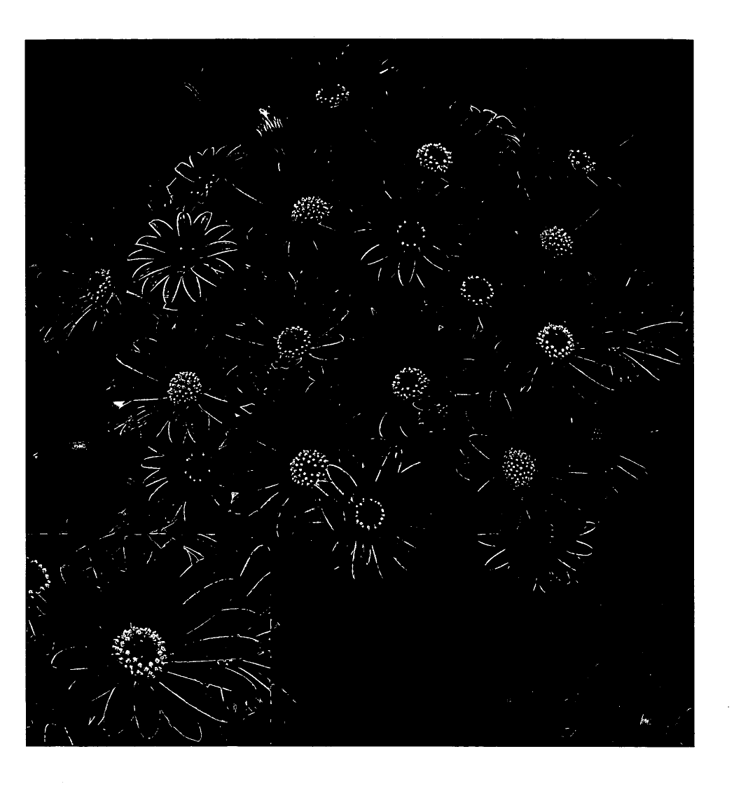

### GENUS AND SPECIES

Osteospermum ecklonis

### VARIETY DENOMINATION

‘Oste Pur’

### BACKGROUND OF THE NEW PLANT

The variety ‘Oste Pur’ originated from a cross made in October, 2000. The female parent is Osteospermum ‘Sunny Martha’ (U.S. Plant Pat. No. PP10,978) which is a light violet plant. The male parent is ‘OX-41, (unpatented) which is a proprietary breeding line. Seed from this cross was sown in June 2001. In October 2001, single-plant selections were made for vegetative propagation using plant habit and flower color as selection criteria. One selection became the new variety ‘Oste Pur’.

‘Oste Pur’ is a product of a planned breeding program intended to create new Osteospermum cultivars with light violet colored flowers, dark green foliage, and vigorous growth.

The new cultivar was created in 2000 in Andjik, The Netherlands and has been asexually reproduced repeatedly by vegetative cuttings and tissue culture in Gilroy, Calif.; and Andijk, The Netherlands over a two ½ year period. The plant has also been trialed at Gilroy, Calif.; and Andijk, The Netherlands. The present invention has been found to retain its distinctive characteristics through successive propagation, and this novelty is firmly fixed.

### DESCRIPTION OF PHOTOGRAPH

This new Osteospermum plant is illustrated by the accompanying photograph which shows blooms, buds, and foliage of the plant in full color, the colors shown being as true as can be reasonably obtained by conventional photographic procedures. Photos were taken from plants grown in The Netherlands in the late winter/spring in a 54° greenhouse. Plants are 4½ months old, had one terminal pinch at 3 weeks old, and are grown in a 4 inch pot with 3 treatments of cycocel.

The photograph shows the mature plant with an inset photo of a close-up of the inflorescence.

### DESCRIPTION OF THE NEW CULTIVAR

The following detailed descriptions set forth the distinctive characteristics of ‘Oste Pur’. The data, which defines these characteristics, were collected from asexual reproductions carried out in Gilroy, Calif. The plant history was taken on 5½ month old plants grown in six inch pots in the late winter/spring season, in a Lexan covered greenhouse under natural light and color readings were taken in the greenhouse under natural lights. Color references are primarily to the RHS Colour Chart of The Royal Horticultural Society of London (RHS) 2001. Texture description details were observed under a dissecting microscope according to The New Royal Horticultural Society Dictionary of Gardening (1992).

### The Plant

Classification: Osteospermum ecklonis.
Botanical: Osteospermum.
Commercial: ‘Oste Pur’.
Form: Upright, semi-compact and mounded.
Growth and branching habit: Good vigorous, freely branched habit.
Height: From soil level to top of blooms: Approximately 40-45 cm.
Width: Approximately 35-45 cm.
Time to produce a finished flowering plant: 8-11 weeks for a 6 inch pot.
Outdoor plant performance: Herbaceous, frost tender perennial patio planters, mixed container plantings or garden beds.
Time to initiate and develop roots: Approximately 8-14 days in the greenhouse.
Root description: Fibrous, well-branched, white.

### The Leaves

Leaf arrangement: Alternate.
Fully expanded, Length: 4.5-4.7 cm; Width: 1.4-1.6 cm.
Leaf shape: Oblanceolate.
Leaf margin: Dentate.
Apex aspect: Broadly acute.
Base aspect: Acuminate.
Leaf color: Upper surface — RHS 137A (green); Under surface RHS 137B (green).
Leaf texture: Pilose.
Venation: Pinnate.
Venation color: RHS 144A (yellow-green).
Petiole length: 1.0 cm.
Petiole diameter: 0.4-0.5 cm.
Petiole color: RHS 144A (yellow-green).
Petiole texture: Pilose.

### The Stem

Length: 30-40 cm.
Diameter: 0.3-0.35 cm.
Stem internode length: 1.5-1.7 cm.
Color: RHS 144A (yellow-green).
Texture: Pilose.
Stem anthocyanin: None.
Peduncle color: RHS 144A (yellow-green).
Peduncle length: 9.0-14.0 cm.
Peduncle diameter: 0.2 cm.
Peduncle texture: Pilose.

### The Bud

Shape: Ovate.
Diameter: 0.7-1.0 cm.
Length: 1.7-1.9 cm.
Color at tight bud (starting to show color): RHS N186C (greyed-purple).

### The Flower

Blooming habit: Plants flower freely from the spring through fall given the proper growing conditions.
Inflorescence/floret type: Terminal and axillary inflorescences held above the foliage. Capitulum inflorescence, radially symmetrical.
Flower (limb) diameter: 5.7-6.2 cm.
Ray petal, Immature Color: Upper surface is RHS N78C (purple) with longitudinal stripes of RHS N78B (purple). Under surface is RHS 186C (greyed-purple) with longitudinal stripes of RHS N78A (purple). Mature Color: Upper surface is RHS N78B (purple) with longitudinal stripes of RHS 78A (purple). Undersurface is RHS 186C (greyed-purple) with longitudinal stripes of RHS N78A.
   Petal length.—2.7-2.8 cm.
   Petal width.—0.7 cm.
   Petal apex shape.—Emarginate to Praemorse.
   Petal base shape.—Cuncate.
   Petal margin.—Entire.
   Petal texture.—Papillose, hirsute on the basal margins.
   Petal quantity.—Approximately 20-25.
Disc petal, tube color: RHS 77C (purple).
   Shape.—Tubular, elongated.
   Length.—0.4 cm.
   Diameter at apex.—0.2-0.3 cm.
   Apex shape.—5-pointed, acute.
   Apex color.—RHS N77A (purple).
   Petal quantity.—Approximately 75-80.
Phyllaries, color: RHS 138A (green).
   Length.—0.8-1.0 cm.
   Width.—0.1 cm.
   Shape.—Linear.
   Apex.—Acute.
   Texture.—Pilose.
   Longevity of individual blooms.—7-14 days.
   Fragrance.—None.

### The Reproductive Organs

Androecium, pollen color: RHS 17A (yellow-orange).
   Filament color.—RHS 76C (purple).
Gynoecium, pistil: 1 per floret.
   Pistil length.—0.4 cm.
   Stigma color.—RHS N77A (purple).
   Style color.—RHS N77A (purple).
   Fruit seed set.—Has not been observed.

### Disease and Insect Resistance

Has not been observed.

### Comparison with Known Cultivars

‘Oste Pur’ differs from the female parent, ‘Sunny Martha’ (U.S. Plant Pat. No. 10,978) in the following ways: ‘Oste Pur’ has a deeper flower color, more compact plant, is earlier to flower and has better branching than ‘Sunny Martha’.

‘Oste Pur’ differs from the male parent, OX-41 in the following ways: ‘Oste Pur’ is later to flower, has larger flowers and has more vigor.

I claim:

1. A new and distinct cultivar of Osteospermum plant as shown and described herein.
